(19) United States
(12) Patent Application Publication
(10) Pub. No.: US 2013/0138055 A1
(43) Pub. Date: May 30, 2013

(54) SPECIALLY-SHAPED BANDAGES FOR TREATING WARTS ON HUMAN DIGITS

(71) Applicant: Curt SAMLASKA, Henderson, NV (US)

(72) Inventor: Curt SAMLASKA, Henderson, NV (US)

(21) Appl. No.: 13/305,700

(22) Filed: Nov. 28, 2011

(51) Int. Cl. A61M 35/00

(52) U.S. Cl. 604/307

(57) ABSTRACT

A bandage is described comprising a rubber-based adhesive and a flexible substrate that is particularly adapted for application onto warts located on the ends of human digits. The flexible substrate is waterproof, skin-colored and shaped in various ways so as to securely attach onto the ends of human fingers and toes. The shape of the bandage promotes long-term use of the bandage to induce the body's inflammatory response and to prevent transmission of the wart virus to other persons.

### FIELD OF THE INVENTION

The present invention relates generally to treating warts, and more specifically the treatment of warts using a polyethylene-backed cloth bandage with a rubber-based adhesive.

### BACKGROUND

Human papillomavirus (HPV) is non-enveloped, meaning that the outer shell or capsid of the virus is not covered by a lipid membrane. Like most non-enveloped viruses, the capsid is geometrically regular and presents icosahedral symmetry. A typical water molecule is less than one nanometer in size—in comparison, a papillomavirus particle is 60 times larger than a water molecule. There are over 150 types of HPVs causing multiple forms of skin infections to include; common warts (Verruca vulgaris), filiform warts, plantar warts, palmar warts, planar or flat warts (Verruca plana), mosaic warts, and genital or venereal warts (Condyloma accuminatum). These mostly benign epithelial tumors are characterized by the formation of thick hyperkeratotic lesions comprised of proliferating wart pulp. Although most human forms of HPV are contracted by direct physical contact from an infected human host, some animal papillomaviruses can be transmitted to humans, such as butcher warts acquired from cattle. HPV infections are very common, and most people will experience infection during their lifetime. HPVs have coexisted with humans for many millennia, and humans are their primary host and reservoir. HPVs are successful pathogens because they evade the human immune response by infecting epidermal cells. The wart proliferates in the epidermal portions of the skin, and never infiltrates the dermis, thus lending itself to a variety of topical treatments. Treatments to include surgical excision, laser vaporization, topical chemical agents (salicylic acid, cantharidin, podophyllin resin, 5-fluorouracil, bleomycin, DNCB, imiquimod, and trichloroacetic acid), and cryotherapy (liquid nitrogen destruction of viral infected epidermal cells).

Cryotherapy is one of the most commonly used topical treatments in the medical community for treating warts. However, freezing warts with liquid nitrogen often results in severe pain and discomfort and is frightening to children and young adults. The fact that none of these forms of treatment have been shown to be better than any other form of treatment underscores the core principle to effective treatment of warts—an immune response must be induced to result in complete resolution of the wart. It has further been shown that the wart virus can lay dormant in the epidermal portions of the skin for up to four years. Recurrence of warts is not uncommon, and can be a lifetime condition requiring periodic intervention. For all of these reasons many dermatologist discourage performing any kind of medical intervention that result in scarring, since the wart can recur in and around the scar after treatment, is not any more effective than non-scarring treatments, and the scarring is not reversible. Thus, there is a need for an easy, aesthetic, and uncomplicated form of treating HPVs.

### SUMMARY

The following presents a simplified summary in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

In one aspect of various exemplary embodiments, an adhesive bandage for treating warts on the ends of human digits is provided, comprising: a flesh-colored multi-sectioned polyethylene first layer, a cloth-based multi-sectioned second layer, wherein the first layer is permanently bonded to the second layer, a polyisoprene-based multi-sectioned adhesive third layer disposed onto the first layer and the second layer, wherein a total thickness of the three layers ranges approximately between 0.23 mm and 0.33 mm, the layers substantially shaped as a cross with four substantially orthogonal sections extending radially from a center of the bandage, and three of the sections being substantially rectangular and the fourth section being substantially semi-circular.

In another aspect of various exemplary embodiments, an adhesive bandage for treating warts on the ends of human digits is provided, comprising: a flesh-colored multi-sectioned polyethylene first layer, a cloth-based multi-sectioned second layer, wherein the first layer is permanently bonded to the second layer, a polyisoprene-based multi-sectioned adhesive third layer disposed onto the first layer and the second layer, wherein a total thickness of the three layers ranges approximately between 0.23 mm and 0.33 mm, the layers substantially shaped as a cross with four substantially orthogonal sections extending radially from a center of the bandage, and two of the sections being substantially rectangular and two of the sections being substantially semi-circular.

In yet another aspect of various exemplary embodiments, an adhesive bandage for treating warts on the ends of human digits is provided, comprising: a flesh-colored multi-sectioned polyethylene first layer, a cloth-based multi-sectioned second layer, wherein the first layer is permanently bonded to the second layer, a polyisoprene-based multi-sectioned adhesive third layer disposed onto the first layer and the second layer, wherein a total thickness of the three layers ranges approximately between 0.23 mm and 0.33 mm, the layers substantially shaped as a rectangle, the rectangle being partially bisected by two lines extending toward the a center of the rectangle from midpoints on opposing edges of the rectangle, each line having a predetermined first length, and each line terminating and intersecting a substantially semi-circular arc of a predetermined second length.

### BRIEF DESCRIPTION OF THE DRAWINGS

The various embodiments can be better understood with reference to the following drawings. Those of skill in the art will understand that the drawings, described below, are for illustrative purposes and are not intended to limit the scope of the present teachings in any way. Accordingly, other embodiments may be used in addition to or instead of the embodiments presented herein, without departing from the spirit and scope of the disclosure. When the same numeral appears in different drawings, it is intended to refer to the same or like components or steps.

FIG. 1 is an illustration of the top plan view of one exemplary adhesive bandage embodiment.

FIG. 2a shows a finger with a periungual wart positioned in the center of the fingertip at the junction of the nail plate and fingertip.

FIG. 2b shows an exemplary adhesive bandage embodiment applied to the finger previously shown in FIG. 2a so as to completely occlude the wart.

FIG. 3 is an illustration of the top plan view of a second exemplary adhesive bandage embodiment.

FIG. 4a shows a finger with a periungual wart positioned in the corner of the nail bed.

FIG. 4b shows an exemplary adhesive bandage embodiment applied to the finger previously shown in FIG. 4a so as to completely occlude the wart.

FIG. 5a shows a finger with a periungual wart positioned in the center of the fingertip at the junction of the nail plate and fingertip.

FIG. 5b shows an exemplary adhesive bandage embodiment applied to the finger previously shown in FIG. 5a so as to completely occlude the wart.

FIG. 6 is an illustration of the top plan view of a third exemplary adhesive bandage embodiment.

FIG. 7a shows a finger with a wart on the distal end of a human digit.

FIG. 7b shows an exemplary adhesive bandage embodiment applied to the finger previously shown in FIG. 7a so as to completely occlude the wart.

### DETAILED DESCRIPTION

Traditional tape, particularly of the type called “duct” tape, has been shown to effectively treat common warts. Various studies, investigations, and comparisons were conducted and results published for the treatment of common warts using “duct tape” therapy. Focht et al. treated common warts in 61 patients with either cryotherapy or duct tape occlusion. After 2 months, 85% of patients in the duct tape group versus 60% in the cryotherapy group had complete resolution of their warts. The authors supplied the patients with industrial or contractor grade duct tape. A subsequent study by de Haen et al. randomized 103 children into one group treated with clear duct tape and a second placebo group treated with corn pads. After 6 weeks the authors concluded there was no statistical difference between the two treatment arms. The study by de Haen et al. has been criticized for (1) using clear duct tape instead of the traditional polyisoprene-based adhesive product and (2) methodological limitations in the study have led colleagues to question the investigator's conclusions. The third investigation was a double-blind controlled clinical trial performed by Wenner et al. comparing clear duct tape and moleskin as a control in 90 patients. With 39 patients in the treatment group and 41 patients in the moleskin control group, the same issues of methodology and statistical limitations that were noted for the study by de Haen et al. would apply. There was no statistical difference between the two treatment groups. Clear duct tape and moleskin both contain an acrylic-based adhesive, whereas standard silver duct tape contains a rubber-based adhesive. Wenner et al. used clear duct tape because they were under the impression that it was the same product as traditional duct tape. It was not until after the study was completed that they discovered the difference. The success of traditional duct tape is associated with the rubber-based adhesive that comes in direct contact with the wart during treatment. The only conclusion one can reach from the two studies using clear duct tape is that the mechanism of action is not related to occlusion or simple debridement.

The actual mechanism for the effectiveness of duct tape in treating warts is unknown. It is believed that the adhesive may provide some therapeutic response, or that the debridement occurs when the tape is removed. Initial studies had the patients apply the duct tape for six days, so it seems likely that debridement alone did not produce such favorable results. Some authors have suggested that the occlusion of the wart “starves it of oxygen” which causes the wart tissue to die. The only problem with this theory is that skin in general does not require exogenous oxygen to survive because the oxygen is supplied through diffusion via capillary tufts within the dermis. There is no “blood supply” in the epidermis at all, and warts have a propensity to elongate the dermal tufts into the expanding wart pulp, thus bringing its own “oxygen supply” along with it. Based on the studies using clear duct tape, it is unlikely that the effectiveness of traditional duct tape is related to simple occlusion or decreased oxygen exposure. What is commonly referred to as “seed warts”—the small black dots often found in warts during debridement—is actually hemorrhage of these capillary tufts producing focal areas of clot within the wart pulp. It is more likely that the polyisoprene-based adhesive (rubber product) and or tachifiers within the adhesive, induces irritation and promotes an inflammatory reaction which eventually results in stimulation of an immune response against the virus. Most topical wart treatments, Compound W, Duofilm, Salicylic Acid, Cantharidin, Podophyllin, Aldara, etc., are designed to produce irritation and inflammation that primarily or secondarily stimulates an immune response.

What is known commercially as duct tape prevents transmission of the wart virus because it is also a waterproof physical barrier that is over four times the thickness of a latex condom. Proof of these claims is evident. In research labs, columns are used to filter various sized particles through filters, including viruses. It is quite obvious that a solution carrying suspended HPVs would be completely blocked if a piece of duct tape was used as a filter interface because it is waterproof. There is no therapeutic product currently available on the market that professes to prevent transmission of wart viruses (condoms are preventative only).

The first adhesive bandages were initially designed by Dickerson after he observed his wife cutting and burning her fingers while cooking. At that time, bandages were sold separately as gauze and tape, but he wanted to make it more manageable for self-application. He cut pieces of adhesive tape and set a piece of gauze in the middle, which he then sterilized. The product was passed on to his employer, Johnson & Johnson® (registered to Johnson & Johnson), who started marketing handmade ones immediately. The first adhesive bandages were not well received and did not gain prominence until 1924 when the first machine manufactured products became available. The central gauze pad is designed to absorb blood and serous fluid from a wound. Most adhesive bandages are perforated and made of cloth with an adhesive applied to secure them in place. They are not designed to be waterproof, but to absorb fluids and blood derivatives while providing the least amount of irritation as possible to the skin, hence the perforations which facilitate evaporation. Anyone who has had an actively bleeding wound knows that blood will infiltrate through an adhesive bandage and soil clothing. A certain degree of protection against viral migration may be possible, but due to the absorptive nature of the product, adhesive bandages are not designed to inhibit viral transmission and, thus, offer no effective prevention of transmission of wart viruses.

Current commercial duct tape is generally sliver or black in color but many other colors and patterns have become available. The standard product comes in rolls 1⅞ inches in width. In using current commercial sources for treating warts there are a number of limitations to include: (1) the color is not cosmetically appealing, and when applied to the skin is conspicuous and unattractive, (2) tearing off a piece, or attempts to cut a piece to cover the wart often produces ragged edges, making it susceptible to lifting off prematurely, (3) patients may end up using clear duct tape or a more traditional looking duct tape that has an acrylic-based adhesive, which does not work as well as duct tape with a rubber-based adhesive, and (4) there are no quality controls in the production of commercial duct tape to insure that contaminants, such as bacteria or fungi, are not introduced into the product. To address these shortcomings, a medical-grade product is disclosed having a variety of specialized pre-cut shapes that lend themselves to treatment of warts on the ends of human digits, including subungual warts and periungual warts, providing sharp adherent edges which are currently not available to the public.

Accordingly, there has been a long-standing need in the medical community for better methods of wart treatment. As detailed below, several wart treatment appliances that are self-administerable are presented.

Many of the related disclosures teach topical treatments for warts using various synthetic or naturally derived solutions. For example, U.S. Pat. No. 5,476,664, granted to Robinson et al., describes one type of occlusion therapy using a composition consisting essentially of an anthralin active ingredient. Another, U.S. Pat. No. 6,593,371, granted to Staggs, discloses a composition and method for treating warts using a naturally derived solution that is applied topically. Yet another, U.S. Pat. No. 5,702,694, granted to Chamness, discloses a composition and method for treating warts using an artificially derived solution. All of these therapies require a second agent, whereas medical grade duct tape occlusion can be used alone as a therapeutic intervention without the use of additional chemicals or solutions.

One related disclosure teaches a system for delivering a drug or topical treatment to affected areas. U.S. Pat. No. 5,641,507, granted to Devillez, describes a delivery system for low-viscosity dermatological ingredients to the skin without the use of repetitive applications. In one embodiment of Devillez's disclosure, a remote application for treating a wart on the heel of a foot is disclosed. Again, however, duct-like tape occlusion therapy does not require chemicals or solutions to be effective.

Yet another disclosure teaches a unique shape for an adhesive bandage that is effective for healing wounds on the ends of human digits. U.S. Pat. No. 5,683,354, granted to Levy, shows an asterisk-shaped adhesive bandage particularly suited for healing wounds on human digits. However, Levy does not disclose the use of duct tape-like material nor does Levy disclose the specialized shapes detailed below.

Of the above related art or products found for sale, none of these approved treatments utilize a duct tape-like material in treating warts. There are publications that claim occlusive articles in various common shapes in order to treat warts. For example, U.S. Patent Application Publication No. 2006/0045897, disclosed by Kennedy, broadly attempts to describe adhesive shapes, adhesive tape, or a kit thereof to provide occlusive articles that can be used on all parts of the body that commonly harbor warts. However, Kennedy does not disclose the use of a duct tape-like material nor the properties thereof for the effective treatment of warts. Furthermore, Kennedy does not disclose any shapes that are effective in occluding warts on the ends of human digits.

U.S. Patent Application Publication No. 2004/0096488, disclosed by Weaver, describes an adhesive bandage with skin-releasable adhesive and perforations on the side portions to allow for easy removal from the skin and to allow the skin to breathe. However, as with Kennedy, Weaver does not disclose embodiments of a bandage that are effective in occluding warts on the ends of human digits.

With the more recent evidence showing the effectiveness of duct tape or duct tape-like material in treating warts there is a need to provide a medical-grade “duct tape” in aesthetic colors and shapes that are conveniently applied as occlusive therapy for the treatment and prevention of transmission of warts on the ends of human digits. As described below, the exemplary embodiments provide a convenient, painless, inexpensive, inconspicuous, and effective treatment of human warts on the ends of human digits.

In the following detailed descriptions of the preferred embodiments, reference is made to the accompanying drawings that form a part hereof, and in which is shown, by way of illustration, specific embodiments in which the invention may be practiced. It is to be understood that other embodiments may be utilized and structural changes may be made without departing from the scope of the present invention.

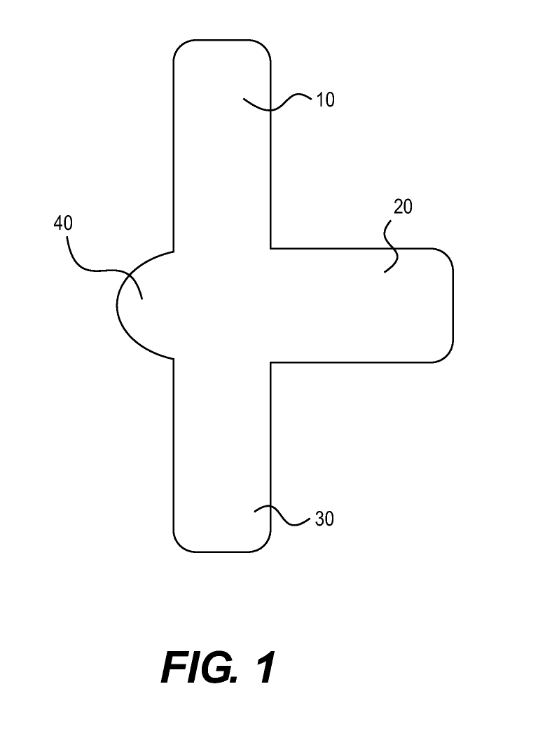

FIG. 1 is an illustration of the top plan view of one preferred embodiment of the present invention adhesive bandage. FIG. 1 shows rectangular sections 10, 20, and 30 semi-circular section 40 of the bandage. The rectangular sections are of a predetermined length that is sufficient to overlap at least one-quarter of the overall length of the bandage once wrapped around a human digit. Generally, the rectangular sections are equal in length. The semi-circular section 40 is particularly adapted to mimic the shape of a human fingernail or toenail which is beneficial because the relatively flat surface of the nail plate and absence of oil or sweat provides a good anchor point and large surface area for adherence of the adhesive on the reverse side of the bandage to the human digit.

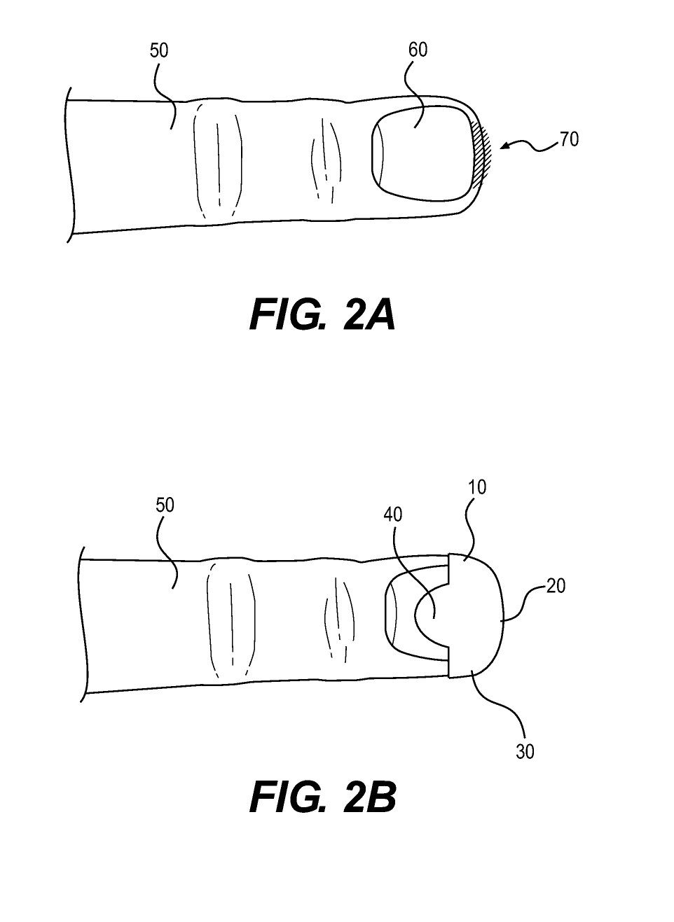

FIG. 2a is an illustration of a person's finger 50 having a periungual wart 70 positioned in the center of the fingertip at the junction of the nail plate 60 and fingertip.

FIG. 2b is an illustration of a person's finger 50 having the first embodiment described in FIG. 1 of the adhesive bandage applied to the finger 50 whereby it completely occludes the wart. The semi-circular section 40 is first applied to the nail plate 60 and the rectangular section 20 is next applied to the underside of the finger 50. Then rectangular sections 10 and 30 are sequentially wrapped around the side of the finger 50 and overlap.

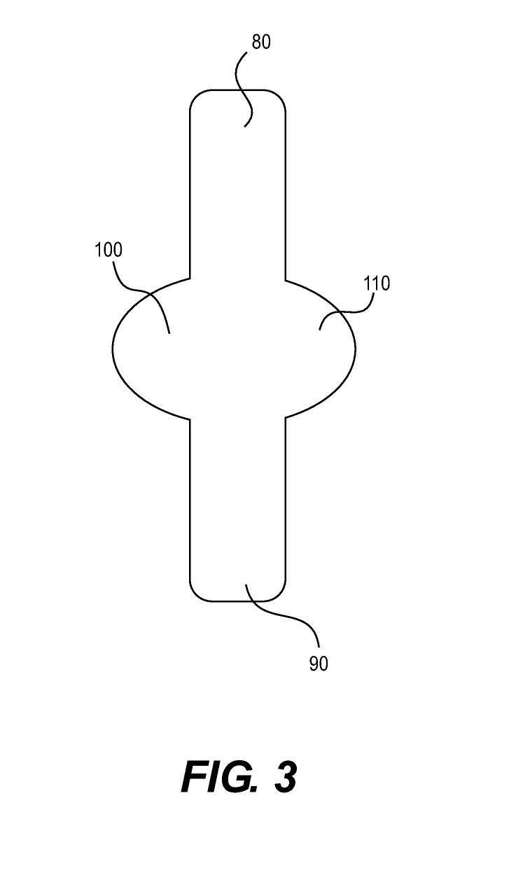

FIG. 3 is an illustration of the top plan view a second exemplary adhesive bandage embodiment. FIG. 3 shows rectangular sections 80 and 90 that are of a predetermined length sufficient to overlap at least one-quarter of the overall length of the bandage once wrapped around a human digit. Generally, the rectangular sections are equal in length. FIG. 3 also shows semi-circular sections 100 and 110 of a predetermined arc length sufficient to fully occlude the wart 70 and have additional circumferential area in which to adhere to the surrounding skin and/or nail plate.

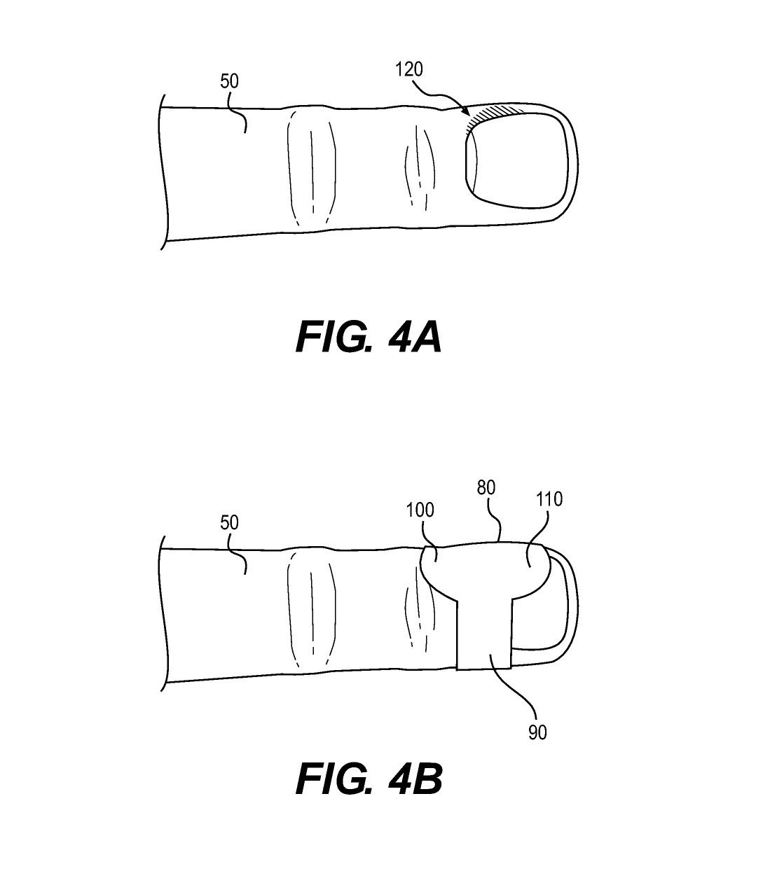

FIG. 4a is an illustration of a person's finger 50 having a periungual wart 120 positioned in the corner of the nail bed.

FIG. 4b is an illustration of a person's finger 50 having the second exemplary embodiment FIG. 3 applied to the finger 50 whereby it completely occludes the wart 120. The semi-circular sections 100 and 110 are first applied to the wart 120. The rectangular sections 80 and 90 are next applied around the sides of the finger 50 and overlap. Finally, semi-circular sections 100 and 110 are sequentially wrapped around the side of the finger 50.

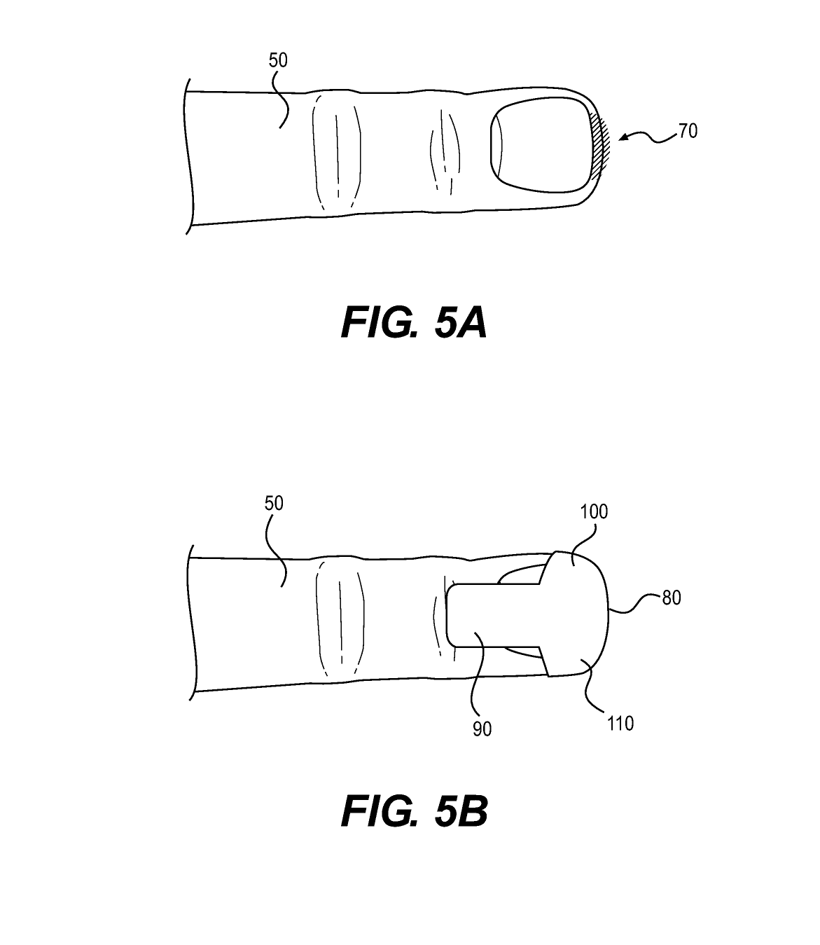

FIG. 5a is an illustration of a person's finger 50 having a periungual wart 70 positioned in the center of the fingertip at the junction of the nail plate 60 and fingertip.

FIG. 5b is an illustration of a person's finger 50 having the second exemplary embodiment FIG. 3 applied to the finger 50 whereby it completely occludes the wart 70. The rectangular section 90 is first applied to the nail plate of the finger 50. Rectangular section 80 is next applied to the underside of the finger 50 by wrapping around the fingertip and the wart 70. Finally, semi-circular sections 100 and 110 are sequentially wrapped around the tip of the finger 50.

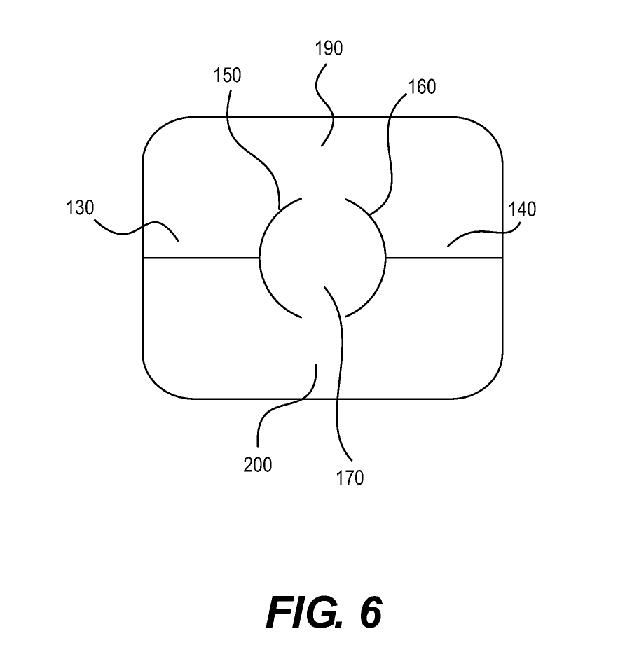

FIG. 6 is an illustration of the top plan view of an exemplary adhesive bandage embodiment. FIG. 6 shows a “rectangle” with rounded corners being partially bisected by lines 130 and 140 extending toward the center of the rectangle 170 from midpoints on opposing edges of the rectangle, each line 130 and 140 having a predetermined length of the rectangle, where each line 130 and 140 does not exceed one-third of the overall length of the rectangle 170, and each line terminating and intersecting semi-circular arcs 150 and 160. Each semi-circular arc 150 and 160 has a maximum length and minimum radius to define a minimum separation between each arc no less than one-eighth of the overall length of the rectangle 170. The lines 130 and 140 and circular arcs 150 and 160 represent cuts that protrude through all three layers of the bandage. These cuts minimize buckling of the bandage during application so as to provide a waterproof barrier to the wart.

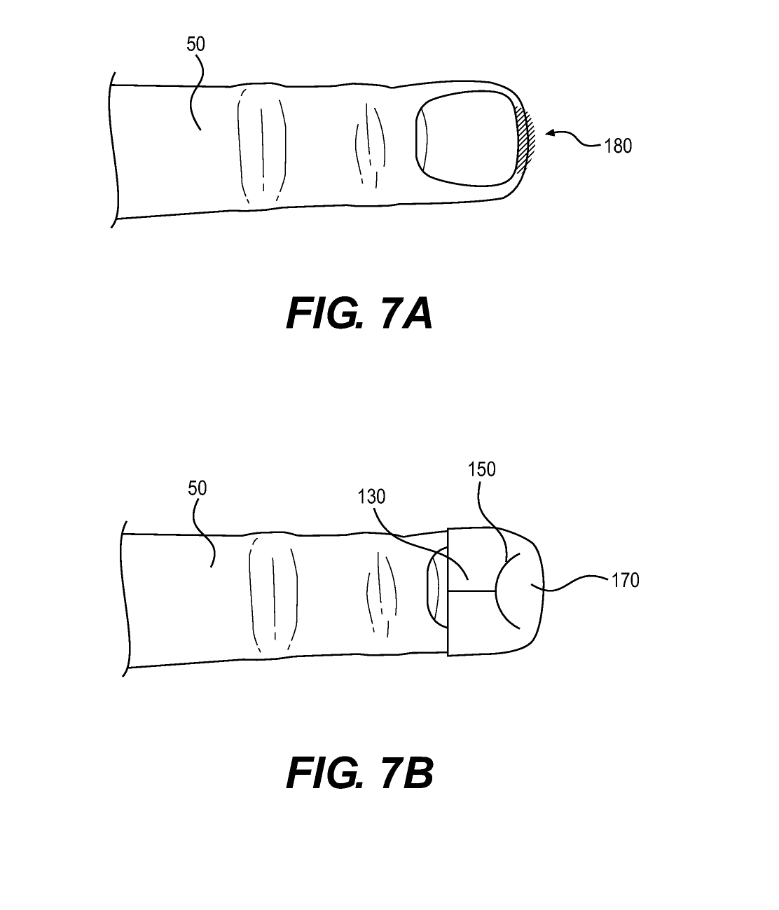

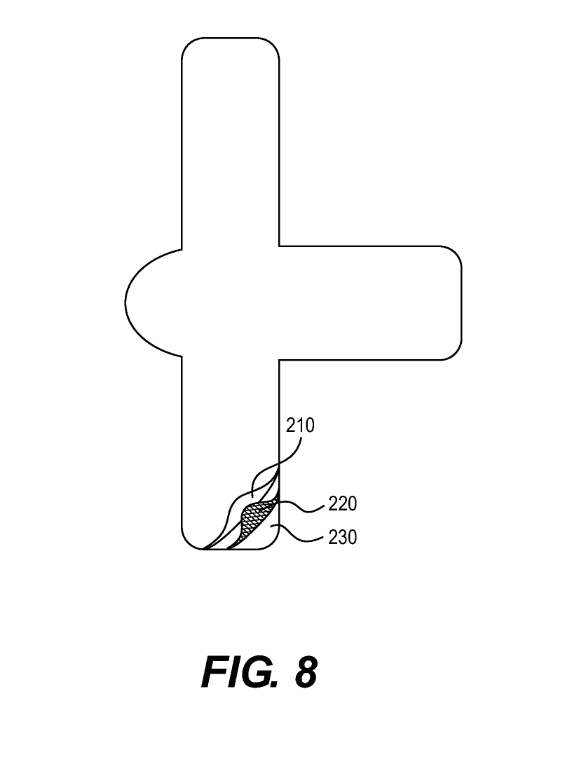

FIG. 7a is an illustration of a person's finger 50 having a wart 180 positioned in the center of the fingertip.

FIG. 7b is an illustration of a person's finger 50 having the exemplary embodiment of FIG. 6 applied to the finger 50 whereby it completely occludes the wart 180. The center section 170 is first applied directly over and centered onto the wart 180. Sections 190 and 200 are next applied to the sides of the finger 50 by wrapping around the fingertip. Then, the sections directly above and below line 130 are sequentially wrapped onto the nail plate 60. Finally, the sections directly above and below line 140 are sequentially wrapped under the finger 50.

FIG. 8 is an illustration of the exemplary embodiment of FIG. 1 showing the polyethylene first layer 210, the cloth-based second layer 220, and the polyisoprene-based adhesive third layer 230.

What has been described above includes examples of one or more embodiments. It is, of course, not possible to describe every conceivable combination of components or methodologies for purposes of describing the aforementioned embodiments, but one of ordinary skill in the art may recognize that many further combinations and permutations of various embodiments are possible. Accordingly, the described embodiments are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An adhesive bandage for treating warts on the ends of human digits, comprising:
a flesh-colored multi-sectioned polyethylene first layer;
a cloth-based multi-sectioned second layer, wherein the first layer is permanently bonded to the second layer; and
a polyisoprene-based multi-sectioned adhesive third layer disposed onto the first layer and the second layer,
wherein a total thickness of the three layers ranges approximately between 0.23 mm and 0.33 mm, the layers substantially shaped as a cross with four substantially orthogonal sections extending radially from a center of the bandage, and three of the sections being substantially rectangular and the fourth section being substantially semi-circular.

2. The adhesive bandage of claim 1, wherein the materials used for the first, second, and third layers are substantially identical to that of industrial or military grade duct tape.

3. An adhesive bandage for treating warts on the ends of human digits, comprising:
a flesh-colored multi-sectioned polyethylene first layer;
a cloth-based multi-sectioned second layer, wherein the first layer is permanently bonded to the second layer; and
a polyisoprene-based multi-sectioned adhesive third layer disposed onto the first layer and the second layer,
wherein a total thickness of the three layers ranges approximately between 0.23 mm and 0.33 mm, the layers substantially shaped as a cross with four substantially orthogonal sections extending radially from a center of the bandage, and two of the sections being substantially rectangular and two of the sections being substantially semi-circular.

4. The adhesive bandage of claim 3, wherein the materials used for the first, second, and third layers are substantially identical to that of industrial or military grade duct tape.

5. An adhesive bandage for treating warts on the ends of human digits, comprising:
a flesh-colored multi-sectioned polyethylene first layer;
a cloth-based multi-sectioned second layer, wherein the first layer is permanently bonded to the second layer; and
a polyisoprene-based multi-sectioned adhesive third layer disposed onto the first layer and the second layer,
wherein a total thickness of the three layers ranges approximately between 0.23 mm and 0.33 mm, the layers substantially shaped as a rectangle, the rectangle being partially bisected by two lines extending toward a center of the rectangle from midpoints on opposing edges of the rectangle, each line having a predetermined first length, and each line terminating and intersecting a substantially semi-circular arc of a predetermined second length.

6. The adhesive bandage of claim 5, wherein the materials used for the first, second, and third layers are substantially identical in all respects to that of industrial or military grade duct tape.
